(19) United States
(12) Patent Application Publication
(10) Pub. No.: US 2004/0131565 A1
(43) Pub. Date: Jul. 8, 2004

(54) Lipstick composition containing essential oils

(76) Inventor: LeTica Lee, New York, NY (US)

Correspondence Address: JAECKLE FLEISCHMANN & MUGEL, LLP, Suite 200, 39 State Street, Rochester, NY, 14610-1310, US

(21) Appl. No.: 10/696,658

(22) Filed: Oct. 29, 2003

Related U.S. Application Data
(60) Provisional application No. 60/422,060, filed on Oct. 29, 2002.

(51) Int. Cl. A61K 7/025; A61K 7/06; A61K 35/78

(52) U.S. Cl. 424/64; 424/74; 424/744; 424/757

(57) ABSTRACT

The present invention provides a lipstick composition that offers both cosmetic and health benefits. Generally, the base lipstick composition is an unscented formula including, but not limited to; candelila, beeswax, aloe vera extract, jojoba oil, titanium dioxide, kaolin, canauba oil, and color with iron oxides, however any known lipstick base formula may be used. In order to provide health benefits, scented essential oils are added to the base lipstick composition. In addition to the above, the present invention also includes 100% pure extract of any fine quality scented herbal plant, flower, or tree containing pure essential scented oil.

This application claims the benefit of U.S. Provisional Patent Application Serial No. 60/422,060, filed Oct. 29, 2002.

### TECHNICAL FIELD

The present invention relates to a cosmetic composition; more particularly, to a lipstick composition; and most particularly to a lipstick composition containing essential oils.

### BACKGROUND

Cosmetics that color lips are extremely popular. Even women who typically do not usually utilize cosmetics, wear lipstick. Known sales of lipstick date back as far as the late 1800's. The type of lipstick offered for sale at that time was a pomade comprising grapefruit mixed with butter and wax. Soon after, additional suppliers entered the market, offering a wide array of lipstick, some of which actually damaged the lips. Beauty writers of the time cautioned women against potential injury of the lips that could be caused by lipstick. As early as the 1920's, it was estimated that 50 million American women used lipstick.

Essential oils have been used for thousands of years in aromatherapy. Generally, the ancient Chinese are credited as the founders of aromatherapy, however it is more likely that man realized the benefits that certain aromatic plants provided some time before that. Furthermore, there are some indications that the ancient Egyptians and ancient Greeks also used aromatic plants to prepare medicinal perfumes.

Generally, essential oils are highly scented droplets that occur in minute quantities in the flowers, stems, leaves, roots and barks of aromatic plants. Additionally, essential oils are complex mixtures of different organic molecules—terpenes, alcohols, esters, aldehydes, ketones and phenols. Synthetic oils are usually made from one or more of the constituents predominant within a particular essential oil; menthol, for example, often substitutes for mint and eucalyptol for eucalyptus. However, it is believed that it is the interaction between each and every component that gives an essential oil its particular character and unique therapeutic properties.

Lipsticks that provide the wearer with benefits beyond the coloring of lips are known. One such lipstick is the Breath Freshening Lipstick, described in U.S. Pat. No. 6,383,475. The lipstick provides a combination of free menthol and a bound menthol substance along with the waxes and usual colorants usually associated in lipstick. The lipstick serves to freshen the breath of the wearer. The aforementioned lipstick serves to mask oral orders, however it does nothing to improve the health and well being of the wearer.

Presently, none of the known lipstick products offer the wearer both cosmetic and health benefits. Additionally, none of the known lipsticks contain essential oils typically associated with aromatherapy.

It would be desirable to provide a lipstick that provides the user with both cosmetic and health benefits.

It would further be desirable to provide a lipstick that was comprised of essential oils thereby providing the user the benefits of aromatherapy.

### BRIEF SUMMARY OF THE INVENTION

The present invention provides a lipstick composition that offers both cosmetic and health benefits. Generally, the base lipstick composition is an unscented formula including, but not limited to; candelila, beeswax, aloe vera extract, jojoba oil, titanium dioxide, kaolin, canauba oil, and color with iron oxides, however any known lipstick base formula may be used. In order to provide health benefits, scented essential oils are added to the base lipstick composition. In addition to the above, the present invention also includes 100% pure extract of any fine quality scented herbal plant, flower, or tree containing pure essential scented oil. While 100% pure essential oil is recommended, other types of essential oils are within the scope of the invention. The essential oils are selected from a group comprising: eucalyptus (decongestant); lavender (respiratory problems, stress); peppermint (headaches, fatigue, shock); rosemary (headaches, fatigue, chest congestion); ylang ylang (anxiety, lowers blood pressure, regulates and alms heart, stress); Bergamot (antidepressant).

### DETAILED DESCRIPTION OF THE INVENTION

The present invention comprises in one form thereof a lipstick composition having essential oils. The essential oils to be added to the base lipstick composition can include, but are not limited to; eucalyptus, lavender, peppermint, rosemary, yang ylang, bergamont, jasmine, apricot and peach, geranium, apple, muguet, and basil.

The preferred embodiment of the present invention includes at least one selected essential oil comprising between 0.001-10.0 percent by weight of the total lipstick composition. The essential oils are to be added to the base lipstick composition. Furthermore, water and oil are excellent conduits for the fragrance of aromatic herbs and extracts. Oil is an important component featured in the production of lipstick products. The wax in lipstick gives it its form and allows it to be packaged and stored safely and conveniently for the consumer. Each (lipstick and aromatic herbs) allows the other to perform its task without interference to the other. Additionally, the base used in the lip wear formula should preferably be unscented so that the addition of the scented essential oil is undisputed in its presence and maximizes the overall effect. Furthermore, the selected essential oils should be 100% pure whenever possible to assure the best results.

An additional embodiment of the present invention is a lipstick composition comprising; prunis dulcis (sweet Almond), apis mellifera (beeswax), palm christi oil, butyrospermun parkii (shea butter), Theobroma cacao (cocoa butter), limnanthes (meadowfoam oil), oenothers biennis (evening primrose), persea americana (avocado) butter, rosa mosqueta (rosehip), aloe barbadensis (aloe) oil, tocopherol (vitamin E), rosmarinus officinalis (rosemary extract), sucrose, azadirachta indica (neem), DMAE, ascorbyl palmitate, salix nigra (black willow bark).

And alternative embodiment of the present invention can comprise petroleum jelly or beeswax and the aforementioned essential oils. The invention provides the same benefits through lip wear as an anti stress formula.

The embodiments described are chosen to provide an illustration of principles of the invention and its practical application to enable thereby one of ordinary skill in the art to utilize the invention in various embodiments and with various modifications as are suited to the particular use contemplated. Therefore, the foregoing description is to be considered exemplary, rather than limiting, and the true scope of the invention is that described in the following claims.

I claim:

1. A lipstick composition comprising:
a lipstick base; and
essential oils.

2. The lipstick composition as in claim 1 wherein said essential oils are, selected from a group consisting of eucalyptus, lavender, peppermint, rosemary, ylang ylang, bergamont, jasmine, apricot and peach, geranium, apple, muguet, and basil.

3. The lipstick composition of claim 1 wherein the essential oils comprise at least about 0.001-10.0 percent by weight of the total lipstick composition.

4. The lipstick composition of claim 1 wherein said lipstick base comprises: candelila, beeswax, aloe vera extract, jojoba oil, titanium dioxide, kaolin, canauba oil, and color with iron oxides.

5. The lipstick composition of claim 1 wherein said lipstick base comprises beeswax.

6. The lipstick composition of claim 1 wherein said lipstick base comprises petroleum jelly.

7. The lipstick composition of claim 1 wherein said essential oils are 100% pure.

8. The lipstick composition of claim 9 wherein said lipstick base is unscented.

9. A lipstick composition comprising: prunis dulcis, apis mellifera, palm christi oil, butyrospermun parkii, theobroma cacao, limnanthes, oenothers biennis, persea americana butter, rosa mosqueta, aloe barbadensis oil, tocopherol, rosmarinus officinalis, sucrose, azadirachta indica, DMAE, ascorbyl palmitate, and salix nigra.

10. A cosmetic formulation comprising:
a base formula; and
essential oils.

11. The cosmetic formulation as in claim 10 wherein said essential oils are selected from a group consisting of eucalyptus, lavender, peppermint, rosemary, ylang ylang, bergamont, jasmine, apricot and peach, geranium, apple, muguet, and basil.

12. The cosmetic formulation of claim 10 wherein the essential oils comprise at least about 0.001-10.0 percent by weight of the total lipstick composition.

13. The lipstick composition of claim 10 wherein said base formula comprises: candelila, beeswax, aloe Vera extract, jojoba oil, titanium dioxide, kaolin, canauba oil, and color with iron oxides.

14. The cosmetic formulation of claim 10 wherein said base formula comprises beeswax.

15. The cosmetic formulation of claim 10 wherein said base formula comprises petroleum jelly.

16. The cosmetic formulation of claim 10 wherein said essential oils are 100% pure.

17. The cosmetic formulation of claim 10 wherein said base formula is unscented.

18. A cosmetic formulation comprising: prunis dulcis, apis mellifera, palm christi oil, butyrospermun parkii, theobroma cacao, limnanthes, oenothers biennis, persea americana butter, rosa mosqueta, aloe barbadensis oil, tocopherol, rosmarinus officinalis, sucrose, azadirachta indica, DMAE, ascorbyl palmitate, and salix nigra.
